(19) United States
(12) Patent Application Publication
(10) Pub. No.: US 2010/0222792 A1
(43) Pub. Date: Sep. 2, 2010

(54) METHOD AND APPARATUS FOR A SOFT TISSUE GRASPING DEVICE

(76) Inventors: Williams S. Barnes, Forsyth, GA (US); Christopher C. Bidwell, Dunwoody, GA (US); George W. Stough, Alpharetta, GA (US)

Correspondence Address: George Stough, 2410 Sandy Creek Farm Rd, Alpharetta, GA, 30004, US

(21) Appl. No.: 12/715,114

(22) Filed: Mar. 1, 2010

Related U.S. Application Data
(60) Provisional application No. 61/208,743, filed on Feb. 27, 2009.

(51) Int. Cl. A61B 17/04

(52) U.S. Cl. 606/148; 606/232

(57) ABSTRACT

The present invention is a novel grasping device for use in handling soft tissue during surgery. The device provides an efficient, time-saving and labor-saving means for compressing and gripping soft tissue, thereby facilitating secure and controlled manipulation of soft tissue during a surgical procedure.

### FIELD OF THE INVENTION

This invention relates to orthopedic surgery in general, and more particularly to methods and apparatus for grasping the ends of a soft tissue graft when preparing it for placement into the body.

### BACKGROUND OF THE INVENTION

A ligament is a piece of fibrous tissue which connects one bone to another, e.g., at a joint. Ligaments are frequently damaged (e.g., detached, torn, ruptured, etc.) as the result of injury and/or accident. A damaged ligament can impede proper motion of a joint and cause pain to the patient.

Various procedures have been developed to repair or replace a damaged ligament. The specific procedures used depend on the particular ligament which is to be restored and the nature and extent of the damage to the ligament.

The anterior cruciate ligament (ACL) of the knee is one ligament which is frequently damaged as the result of injury and/or accident. A damaged ACL can cause instability of the knee, may lead to arthritis and/or damage to other bodily structures, and can cause substantial pain for the patient.

Numerous procedures have been developed to replace a badly damaged ACL through a graft ligament replacement procedure. These ACL replacement procedures generally involve drilling a bone tunnel across the tibia and up into the lower portion of the femur. Then a graft ligament, consisting of a harvested ligament or tendon or an artificial ligament, is prepared by the surgeon by suturing the distal end(s) and leaving towing sutures trailing from it. The distal end is then passed through the tibial portion of the bone tunnel, across the interior of the joint, and up into the femoral portion of the bone tunnel. Then the distal portion of the graft ligament is secured in the femoral tunnel and the proximal end of the graft ligament is pulled taut and secured in the tibial tunnel. Once secured, the graft's end is severed at the tibia and discarded.

The preparation of the graft end(s) generally requires the surgeon to whip stitch the graft in such a manner that the tissue can be pulled taut without tearing the tissue and so that the suture leaves a proper length of graft material for pulling during final fixation in the tibial tunnel. This takes valuable surgical time, during which the patient is under anesthesia and in tourniquet. These two factors, in addition to the high financial cost of the surgeon's time, O.R. time, staff time, etc., make it highly desirable to keep the surgery time to a minimum whenever reasonably possible. Therefore, time is clearly of the essence during surgery.

### SUMMARY OF THE INVENTION

The present invention provides a new method and apparatus for quickly, securely and easily grasping the ends of a soft tissue graft for purposes of handling the graft.

More particularly, the present invention provides a new method and apparatus for grasping and pulling the soft tissue graft while the surgeon is affixing said graft in the tibial tunnel during ACL reconstruction.

The present invention may also be used in other types of orthopedic surgery where soft tissue grafts other than ACL grafts must be pulled and/or handled.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

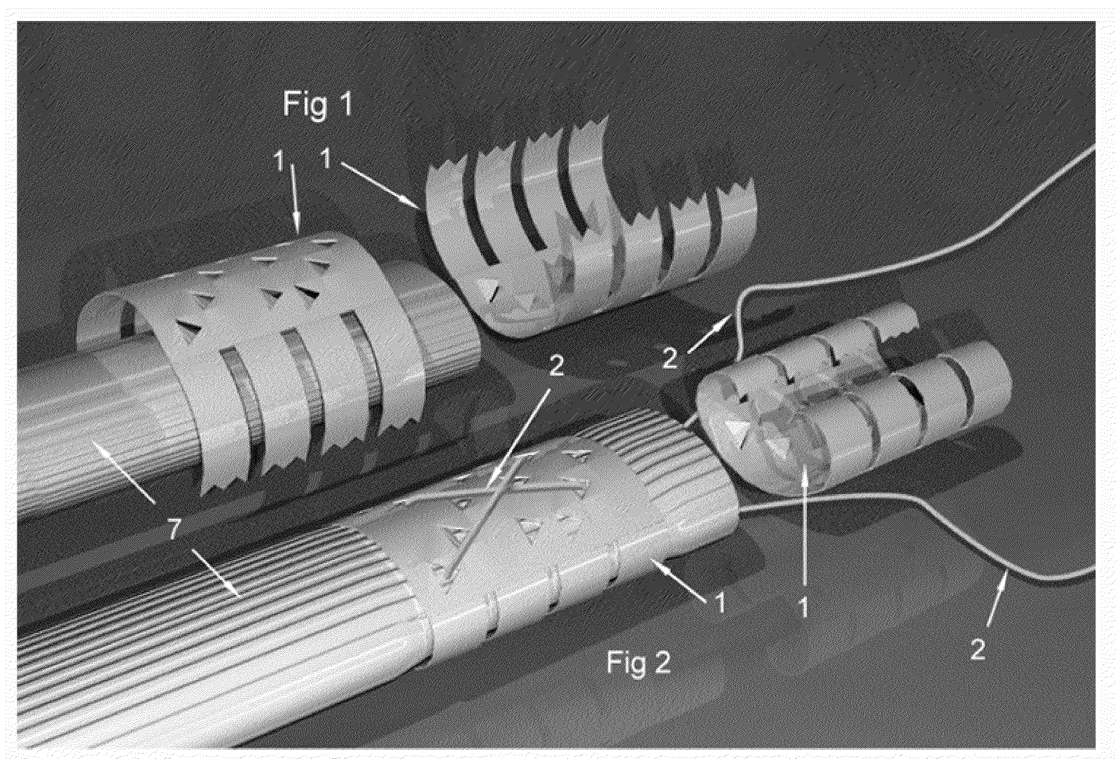

(1) On page 1, FIGS. 1 and 2 are schematic perspective views showing a soft tissue grasping device in the prior to crimping and crimped formations respectively.

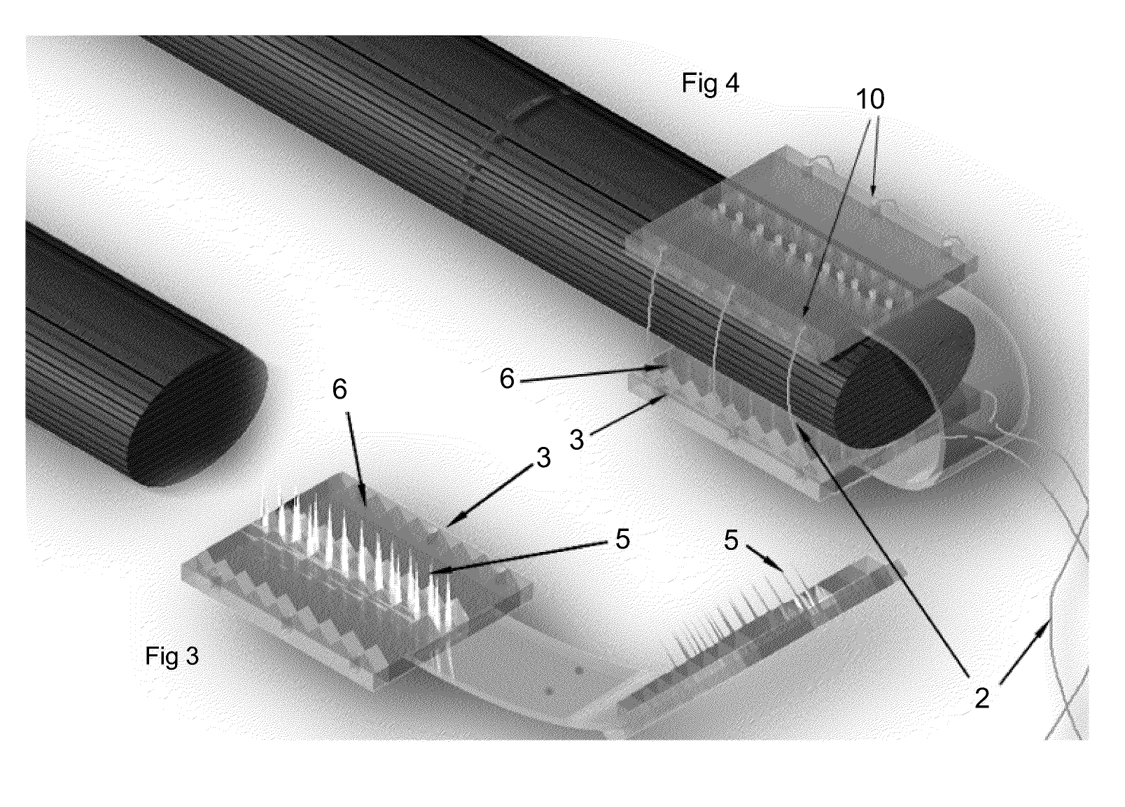

(2) On page 2, FIGS. 3 and 4 are schematic perspective views showing a soft tissue grasping device in the open (with tendon out of device) and closed (with tendon in the device) positions respectively.

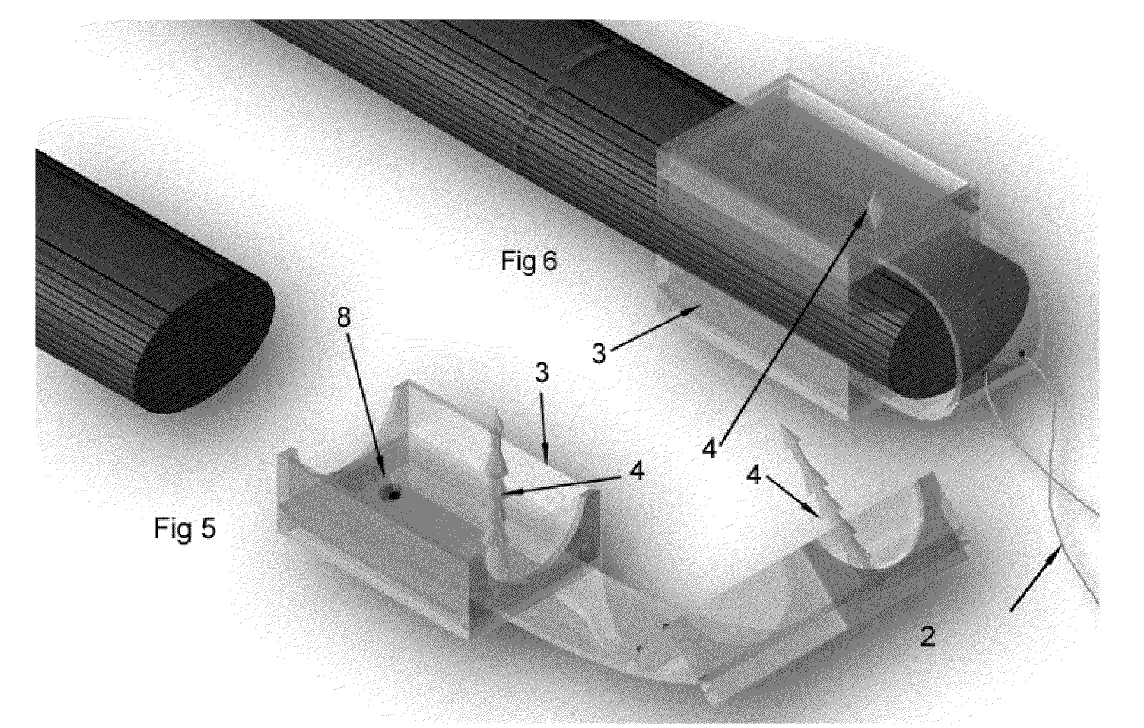

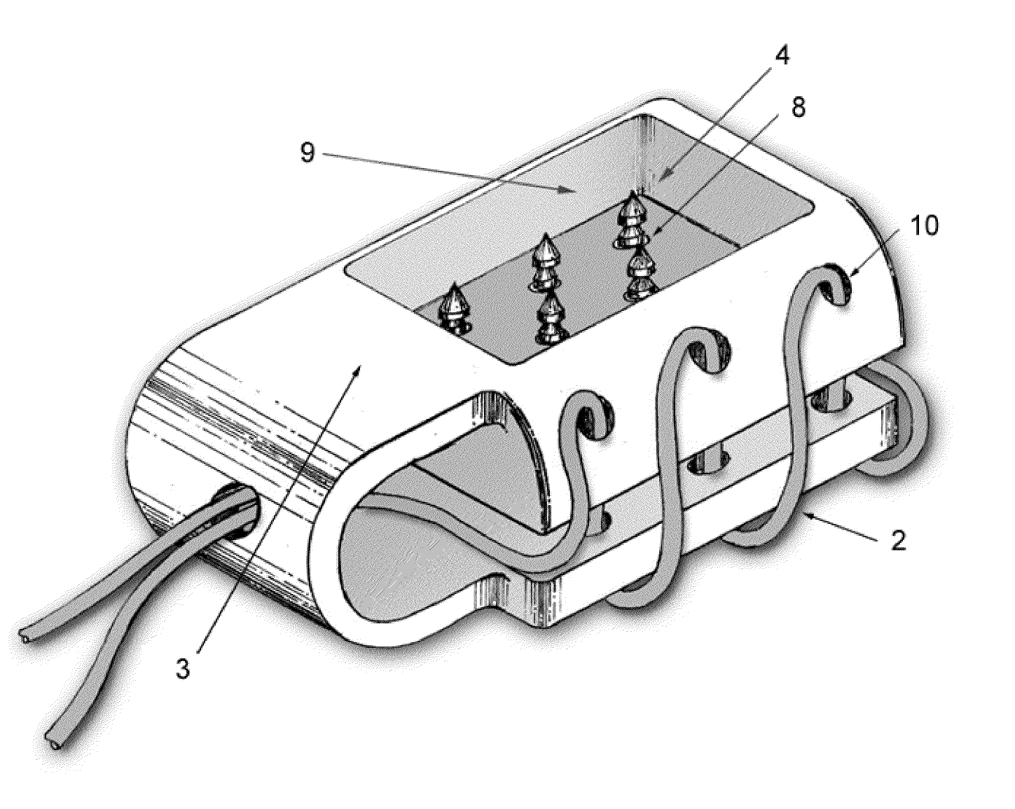

(3) On page 3, FIGS. 5 and 6 are schematic perspective views showing a soft tissue grasping device in the open (with tendon out of device) and closed (with tendon in the device) positions respectively.

(4) On Page 4 is a schematic perspective view showing a soft tissue grasping device in the closed position, but with no tendon graft in place.

### DETAILED DESCRIPTION OF THE PREFERRED EMBODIMENTS

The present invention provides a new method and apparatus for quickly, securely and easily grasping the ends of a soft tissue graft for purposes of handling the graft. For convenience, the present invention will hereinafter be discussed in the context of its use for ACL reconstruction, using a harvested autograft, a donated allograft or an artificial graft.

(1) On page 1, FIGS. 1 and 2 are schematic perspective views showing a one-piece soft tissue grasping device in accordance with the present invention. Tendon graft 3 is laid through the grasping device 1 in its non-crimped state. FIG. 2 shows a tendon graft with a crimped grasping device—performed with a tool designed especially for that purpose—securely wrapped around it and piercing the graft tissue on the underside. Suture material 2 is shown further securing the tissue to the grasping device and sewn in such a way as to leave towing sutures trailing from the proximal end of the graft. In some instances said grasping device may be used on both ends of the graft, leaving towing sutures trailing from the distal and proximal ends of the graft.

(2) On page 2, FIGS. 3 and 4 are schematic perspective views showing a two-piece soft tissue grasping device connected with a living hinge in accordance with the present invention. The interior surfaces are covered with gripping textures, ridges 6 and spiked “teeth” 5 to secure the graft tissue. Suture material may be then threaded from hole to hole and around or through the graft tissue to further secure it, and then out the proximal end to form towing sutures.

(3) On page 3, FIGS. 5 and 6 are schematic perspective views showing a two-piece soft tissue grasping device connected with a living hinge in accordance with the present invention. The two halves of the device 3 are brought together and the integral barbed prongs 4 pierce the tissue and the heads of the prongs are forced through corresponding receiving holes 8 to lock the halves together. Suture material is threaded through the holes on the proximal end to create towing sutures.

(4) On Page 4 is a schematic perspective of a soft tissue grasping device in the closed position, showing a multiplicity of barbed prongs 4 and showing a recessed area 9 which will protect the protruding prongs once received by the receiving holes 8. Sutures 2 are shown threaded through the suture holes 10, giving the device additional security and clamping ability. Suture holes 10 would appear on both sides of the device and on the proximal end to accept the suture ends (towing sutures).

### Some Aspects of the Invention

The soft tissue grasping device is comprised of upper and lower clamping formations which come together around the tissue and mate in such a way as to firmly compress the tissue.

The soft tissue grasping device's upper and lower formations may be connected with a living hinge and operate in a “clamshell” manner.

The soft tissue grasping device may be comprised of a single formation which is bent or otherwise deformed to grasp the soft tissue.

The soft tissue grasping device may incorporate holes at its distal end for introduction of towing suture to stretch the assembled graft ligament before fixing it in the tibial tunnel.

The soft tissue grasping device may have a multiplicity of pores or openings to accept suture material to additionally secure the graft ligament and create towing sutures.

The soft tissue grasping device may incorporate integral towing suture material already inserted within the device.

The soft tissue grasping device may incorporate gripping “teeth”, stabbing protrusions, serrations, prongs, etc. that will provide increased means for securing the graft ligament.

The soft tissue grasping device may have prongs integrally formed or separately inserted to pierce the soft tissue and affix it within the device.

The soft tissue grasping device may be produced from various polymers, or other materials (e.g., metals, ceramic, etc.)or a combination of those materials.

The soft tissue grasping device may be comprised of two mating halves that are brought together to form a single element.

The soft tissue grasping device may be sized to fit all grafts or may come in specific sizes.

The soft tissue grasping device system may include a tool to deform the device tightly around, through or into the tissue or to force the halves together.

The soft tissue grasping device system may include a tool to deform, press or otherwise secure the device to the graft, to insert fastening sutures and to add towing sutures to the assembly.

The system may include a stand to facilitate the deforming, clamping, pressing, or otherwise securing the device to the graft and to insert fastening sutures and/or to add towing sutures to the assembly.

### A Preferred Method of Use

1. The end of a ligament graft is laid loosely onto or in or through the grasping device.

2. The grasping device is clamped onto the end of the of the soft tissue graft, manually or by using a tool.

2. Sutures are threaded through the grasping device and the graft end to make a consolidated unit, leaving a length of suture trailing from the graft as towing suture.

### Some Advantages/Benefits of the Present Invention

The soft tissue grasping device provides a quick, simple and secure method of attaching towing sutures to a soft tissue graft, resulting in a stable and secure assembly requiring considerably less surgical time while the patient is under anesthesia and in tourniquet.

### Modifications

It will be understood that many additional changes in the details, materials, steps and arrangements of parts, which have been herein described and illustrated in order to explain the nature of the invention, may be made by those skilled in the art while remaining within the principles and scope of the present invention.

What is claimed is:

1. Apparatus for use in securing end(s) of soft tissue for handling and stretching soft tissue during orthopedic surgery, the apparatus comprising:
A single formation which is bent or otherwise deformed to wrap around and firmly compress and grip the soft tissue.

2. Apparatus according to claim one further comprising holes at its distal end for introduction of towing suture to pull and thereby stretch the soft tissue.

3. Apparatus according to claim one further comprising a multiplicity of pores or openings to accept suture material to additionally secure the graft ligament and create towing sutures.

4. Apparatus according to claim one further comprising integral towing suture material already inserted within the device.

5. Apparatus according to claim one further comprising gripping “teeth”, stabbing protrusions, serrations, prongs, etc. that will provide increased means for securing the graft ligament.

6. Apparatus according to claim one further comprising a tool to deform, press or otherwise secure the device to the graft, to insert fastening sutures and to add towing sutures to the assembly.

7. Apparatus according to claim one further comprising a stand to facilitate the deforming, clamping, pressing, or otherwise securing the device to the graft and to insert fastening sutures and/or to add towing sutures to the assembly.

8. Apparatus according to claim one further comprising hole(s) or protrusion(s) to allow an additional tool or device to couple with said grasping device to facilitate pulling.

9. Apparatus for use in securing end(s) of soft tissue for handling and stretching soft tissue during orthopedic surgery, the apparatus comprising:
Upper and lower clamping formations which come together around the tissue and mate in such a way as to firmly compress the tissue.

10. Apparatus according to claim nine further comprising a living hinge connecting the upper and lower formations so it will operate in a “clamshell” manner.

11. Apparatus according to claim nine further comprising holes at its distal end for introduction of towing suture to stretch the assembled graft ligament before fixing it in the tibial tunnel.

12. Apparatus according to claim nine further comprising a multiplicity of pores or openings to accept suture material to additionally secure the graft ligament and create towing sutures.

13. Apparatus according to claim nine further comprising integral towing suture material already inserted within the device.

14. Apparatus according to claim nine further comprising gripping “teeth”, stabbing protrusions, serrations, prongs, etc. that will provide increased means for securing the graft ligament.

15. Apparatus according to claim nine further comprising a tool to deform, press or otherwise secure the device to the graft, to insert fastening sutures and to add towing sutures to the assembly.

16. Apparatus according to claim nine further comprising a stand to facilitate the deforming, clamping, pressing, or otherwise securing the device to the graft and to insert fastening sutures and/or to add towing sutures to the assembly.

17. Apparatus according to claim nine further comprising hole(s) or protrusion(s) to allow an additional tool or device to couple with said grasping device to facilitate pulling.
